(19) United States
(12) Patent Application Publication
(10) Pub. No.: US 2024/0026469 A1
(43) Pub. Date: Jan. 25, 2024

(54) Methods for Microbial Identification in Clinical Specimens by Differential Ribosomal RNA Probe Hybridization

(71) Applicants: MicrobeDX, Inc., Pacific Palisades, CA (US); The Regents of the University of California, Oakland, CA (US)

(72) Inventors: Bernard Churchill, Los Angeles, CA (US); Scott Adam Churchman, Santa Monica, CA (US); David Arnold Haake, Culver City, CA (US); Colin Wynn Halford, Los Angeles, CA (US); Roger Knauf, Cincinnati, OH (US); Gabriel Monti, Cypress, CA (US)

(21) Appl. No.: 18/468,971

(22) Filed: Sep. 18, 2023

Related U.S. Application Data
(63) Continuation of application No. 17/054,293, filed on Nov. 10, 2020.

(51) Int. Cl. C12Q 1/689; C12Q 1/6806

(52) U.S. Cl. CPC C12Q 1/689; C12Q 2600/158; C12Q 1/6806

(57) ABSTRACT

A method of identifying a target microbe in a specimen and including the steps of a) obtaining a specimen, b) lysing the specimen to release a plurality of rRNA molecules from one or more first target microbes in the specimen, c) contacting the specimen with a plurality of first oligonucleotide probe sets configured to selectively bind to rRNA molecules released from the target microbe thereby forming a plurality of first hybridized complexes, each first hybridized complex including one first capture probe and one first detector probe and one of the plurality of rRNA molecules, and d) analyzing the first hybridized complexes to identify a first target microbe in the specimen.

### CROSS REFERENCE TO RELATED APPLICATION

The present application is a continuation application of U.S. patent application Ser. No. 17/054,293. filed on Nov. 10, 2020, which is a National Stage application, filed under section 371, of International Patent Application No. PCT/US2019/032227, filed May 14, 2019, which claims the benefit under 35 U.S.C. § 119(e) of Provisional Patent Application No. 62/671,389, filed on May 14, 2018 and entitled Methods for Microbial Identification in Clinical Specimens by Differential Ribosomal RNA Probe Hybridization, the entirety of which is hereby incorporated by reference.

This application incorporates by reference a Sequence Listing in computer readable form (CRF) as a text file entitled “64092US01 SeqList For Filing” created on Jul. 16, 2021 and having a size of 18,000 bytes.

### FIELD OF THE INVENTION

The present invention relates to a method for microbial identification. More specifically, the invention relates to a method for microbial identification in a clinical specimen based on detection of ribosomal RNA.

### SUMMARY

This summary is intended to introduce the reader to the more detailed description that follows and not to limit or define any claimed or as yet unclaimed invention. One or more inventions may reside in any combination or sub-combination of the elements or process steps disclosed in any part of this document including its claims and figures.

Some methods of microbial identification based on detection of ribosomal RNA (“rRNA”) are known in the art. For example, Liao et al. (2006)¹ and Liao et al. (2007)² describe methods of microbial identification in clinical specimens. Such methods of microbial identification based on detection of rRNA include the following four steps: 1) Lysis to release rRNA; 2) Neutralization; 3) Hybridization of target rRNA with a capture probe and detector probe; and 4) Detection of capture probe—target rRNA—detector probe complexes. Liao et al. (2006) also describes performing an rRNA detection assay directly on urine specimens from patients with a urinary tract infection to identify the bacteria in the clinical specimen. ¹Liao J C, Mastali M, Gau V, Suchard M A, Moller A K, Bruckner D A, Babbitt J T, Li Y, Gornbein J, Landaw E M, McCabe E R B, Churchill B M, Haake D A. Use of electrochemical DNA biosensors for rapid molecular identification of uropathogens in clinical urine specimens. J Clin Microbiol. 2006; 44(2):561-70.²Liao J C, Mastali M, Li Y, Gau V, Suchard M, Babbitt J T, Gornbein J, Landaw E M, McCabe E R, Churchill B M, Haake D A. Development of an advanced electrochemical DNA biosensor for bacterial pathogen detection. J Mol Diagn. 2007; 9:158-68.

Species-specific microbial identification may be accomplished by using one or more probes on a clinical specimen which has been lysed to expose the rRNA of the target microbe(s). A probe set comprising a capture probe and a detector probe, each comprising an oligonucleotide, may hybridize with a specific target sequence of the rRNA of a target species. The presence of hybridized detector probe which is detectably labelled may be determined using a suitable detection means. Some examples of species-specific oligonucleotide probes are known in the art, for example as described in Liao (2006) and U.S. Pat. No. 7,763,426 to Haake et al.

However, oligonucleotide probes known in the art may have relatively lower specificity and reliability, and may not be optimized to detect microbial species in clinical specimens from patients suspected of having certain infections, such as urinary tract infection.

Microbial identification may be achieved more rapidly by applying a mixture of a plurality of species-specific probes to a clinical specimen. However, the presence of multiple distinct species-specific probes may increase the risk of interference between the probes as well as between probes and target rRNA thereby reducing the reliability of results.

It is an object of the present invention to provide a novel method for identifying microbial species in a clinical specimen.

It is another object of the present invention to provide a novel method for identifying microbial species in a clinical specimen using oligonucleotide probes.

It is another object of the present invention to provide a novel method for identifying microbial species in a clinical specimen using oligonucleotide probes, wherein the microbial species includes both gram-positive and gram-negative bacteria and fungi.

It is another object of the present invention to provide a novel method for identifying microbial species in a clinical specimen using oligonucleotide probes, wherein the probes hybridize with target rRNA sequences of the microbial species.

It is another object of the present invention to provide a novel method for identifying one or more microbial species using species-specific oligonucleotide probes in a clinical specimen containing an unknown number of microbial species.

It is another object of the present invention to provide a novel method for identifying one or more microbial species in a single sample of clinical specimen using a medium containing a plurality of species-specific oligonucleotide probes.

It is another object of the present invention to provide a novel method for identifying one or more microbial species in a single sample of clinical specimen using a medium containing a plurality of oligonucleotide probes wherein each oligonucleotide probe is adapted to hybridize with a distinct target rRNA sequence.

It is another object of the present invention to provide a novel method for identifying one or more microbial species in a single sample of clinical specimen using a medium containing a plurality of oligonucleotide probes, wherein some probes are adapted to detect a microbial family, while other probes are adapted to detect a particular microbial genus and other probes are adapted to a particular microbial species.

It is another object of the present invention to provide a novel method for identifying one or more microbial species in a clinical specimen, wherein the identification of microbial species may then be used for application in a clinical setting.

It is another object of the present invention to provide a novel method for identifying one or more microbial species in a clinical specimen, wherein the microbial identification may then be used to determine the likelihood of microbial infection.

It is another object of the present invention to provide a novel method for identifying one or more microbial species in a clinical specimen, wherein the microbial identification may then be used to optimize the clinical specimen for antimicrobial susceptibility testing.

It is another object of the present invention to provide a novel method for identifying one or more microbial species in a clinical specimen, wherein the identification of microbial species may be sufficiently accurate for further clinical actions within a clinically acceptable time limit.

It is another object of the present invention to provide a novel method for identifying one or more microbial species in a clinical specimen, wherein the identification of microbial species may be completed in less than two hours of obtaining the clinical specimen.

It is another object of the present invention to provide a novel method for identifying one or more microbial species in a clinical specimen, wherein the identification of microbial species may be completed in less than one hour of obtaining the clinical specimen.

It is another object of the present invention to provide a novel method for identifying one or more microbial species in a clinical specimen, wherein the identification of microbial species may be completed in less than 30 minutes of obtaining the clinical specimen.

It is another object of the present invention to provide a novel method for identifying one or more microbial species in a clinical specimen, wherein the identification of microbial species may be used to determine clinical treatment for urinary tract infection (UTI) or sepsis via blood culturing.

Accordingly, an aspect of the present invention provides a method of identifying a first target microbe in a specimen, the method comprising:
(a) lysing the specimen to release rRNA molecules from the first target microbe thereby producing a lysate;
(b) contacting the rRNA molecules with a first oligonucleotide probe set configured to selectively bind to the rRNA molecules; each first oligonucleotide probe set comprising a first capture probe and a first detector probe, thereby forming a first hybridized complex, the first hybridized complex comprising rRNA molecules bound to the first oligonucleotide probe set; and
(c) analyzing the first hybridized complex to identify the first target microbe.

Another aspect of the present invention provides a method of identifying a first target microbe in a specimen, the method comprising:
(a) lysing the specimen to release rRNA molecules from the first target microbe thereby producing a lysate;
(b) contacting the rRNA molecules with a first oligonucleotide probe set configured to selectively bind to the rRNA molecules; each first oligonucleotide probe set comprising a first capture probe and a first detector probe, thereby forming a first hybridized complex, the first hybridized complex comprising rRNA molecules bound to the first oligonucleotide probe set;
(c) analyzing the first hybridized complex to identify the first target microbe;
(d) contacting the rRNA molecules with a second oligonucleotide probe set configured to selectively bind to rRNA molecules released from the second target microbe; the second oligonucleotide probe set comprising a second capture probe and second detector probe, thereby forming a second hybridized complex, the hybridized complex comprising rRNA molecules bound to the second oligonucleotide probe set; and
(e) analyzing the second hybridized complexes to identify the second target microbe.

Another aspect of the present invention provides an oligonucleotide probe set for use in identifying a first target microbe in a specimen, wherein the oligonucleotide probe set comprises a capture probe and a detector probe, the capture probe and detector probe each being adapted to selectively hybridize to a first target sequence of rRNA molecules released from the first target microbe.

Another aspect of the present invention provides oligonucleotide probe sets comprising a capture probe and a detector probe. In some embodiments, the probe sets of the present invention may further comprise a helper probe.

Another of the present invention provides a probe panel disposed on a substrate, the probe panel comprising a plurality of detection regions, each detection region comprising an oligonucleotide probe set configured to selectively bind to RNA molecules released from a pre-determined microorganism, each detection region comprising a different oligonucleotide probe set.

Another of the present invention provides a system for identifying at least a first and second target microbes in a specimen, the system comprising:
(a) probe panel; and
(b) a detection apparatus configured to detect hybridized complexes present in any detection regions of probe panel.

These and other aspects will become apparent to those of skill in the art upon reviewing the present specification.

### BRIEF DESCRIPTION OF THE DRAWINGS

Embodiments of the present invention will be described with reference to the accompanying drawings, in which:

FIG. 1, in a flowchart, illustrates the steps involved in identifying microbial species in a clinical specimen using oligonucleotide probes which may hybridize with target rRNA sequences in one or more microbial species;

FIG. 2 schematically illustrates an example of rRNA hybridization with a pair of oligonucleotide probes comprising a capture probe and a detector probe;

FIG. 3 schematically illustrates one example of a probe panel configured to detect/identify 13 target microbes;

FIG. 4, in a graph, illustrates the species-specific detection of certain microbial species in a representative clinical specimen;

FIG. 5, in a graph, illustrates the detection sensitivity for E. coli cells using an embodiment of the present invention in a representative clinical specimen; and

FIG. 6 schematically illustrates another example of a probe panel configured to detect/identify one or more target microbes; and

FIG. 7 schematically illustrates one example of a system for identifying at least a first and second target microbes in a specimen.

### DETAILED DESCRIPTION

The present invention relates to a method of identifying a first target microbe in a specimen, the method comprising: (a) lysing the specimen to release rRNA molecules from the first target microbe thereby producing a lysate; (b) contacting the rRNA molecules with a first oligonucleotide probe set configured to selectively bind to the rRNA molecules; each first oligonucleotide probe set comprising a first capture probe and a first detector probe, thereby forming a first hybridized complex, the first hybridized complex comprising rRNA molecules bound to the first oligonucleotide probe set; and (c) analyzing the first hybridized complex to identify the first target microbe.

Preferred embodiments of this method may include any one or a combination of any two or more of any of the following features:
the first capture probe comprises an oligonucleotide adapted to hybridize with a first target sequence of the rRNA molecules released in step (a); and wherein the first detector probe comprises a detectably labeled oligonucleotide adapted to hybridize with a second sequence of the rRNA molecules;
the method includes a further step the further step of neutralizing the lysate of the specimen produced in step (a);
the method comprising the further step of washing away unhybridized first detector probes and/or unhybridized first capture probes from step (b).
the oligonucleotide probe sets further comprise a helper probe, wherein the helper probes include sequences adapted to hybridize with a helper sequence on the capture probe;
step (b) comprises contacting the rRNA molecules with a panel comprising a plurality of oligonucleotide probe sets, each probe set adapted to selectively bind to rRNA molecules released from a pre-determined microorganism;
the microorganism is selected from the group consisting of: Escherichia coli (EC), Klebsiella pneumoniae (KP), Proteus mirabilis (PM), Pseudomonas aeruginosa (PA), Staphylococcus saprophyticus (SS), Staphylococcus aureus (SA), Streptococcus agalactiae (GB), Enterococcus faecium (EM), Candida albicans (CA), Enterobacteriaceae (EB), Enterococci (EF), Candida species (CN), Citrobacter freundii (CF), Serratia marcescens (SM), Enterobacter cloacae (EL), Enterobacter hormaechei (EH), and Klebsiella oxytoca (KX), Klebsiella aerogenes (KA), Morganella morganii (MM), Acinetobacter baumanii (AB), Streptococcus pyogenes (SY), Streptococcus pneumoniae (SP), Streptococcus viridans (SV), Stenotrophomonas maltophilia (XM), Staphylococcus epidermidis (SE);
the plurality of first oligonucleotide probe sets configured to selectively bind to 16S rRNA sequences, or 23S rRNA sequences;
the method further comprises identifying a second target microbe in the specimen by including the steps of: (d) contacting the rRNA molecules with a second oligonucleotide probe set configured to selectively bind to rRNA molecules released from the second target microbe; the second oligonucleotide probe set comprising a second capture probe and second detector probe, thereby forming a second hybridized complex, the hybridized complex comprising rRNA molecules bound to the second oligonucleotide probe set; and (e) analyzing the second hybridized complexes to identify the second target microbe.
steps (d) and (e) are performed simultaneously with steps (b) and (c);
the first and second oligonucleotide probe sets are configured to selectively bind to rRNA molecules released from different pre-determined microorganisms;
the second oligonucleotide probe set is configured to selectively bind to rRNA molecules released from a microorganism selected from the group consisting of: Escherichia coli (EC), Klebsiella pneumoniae (KP), Proteus mirabilis (PM), Pseudomonas aeruginosa (PA), Staphylococcus saprophyticus (S S), Staphylococcus aureus (SA), Streptococcus agalactiae (GB), Enterococcus faecium (EM), Candida albicans (CA), Enterobacteriaceae (EB), Enterococci (EF), Candida species (CN), Citrobacter freundii (CF), Serratia marcescens (SM), Enterobacter cloacae (EL), Enterobacter hormaechei (EH), and Klebsiella oxytoca (KX), Klebsiella aerogenes (KA), Morganella morganii (MM), Acinetobacter baumanii (AB), Streptococcus pyogenes (SY), Streptococcus pneumoniae (SP), Streptococcus viridans (SV), Stenotrophomonas maltophilia (XM), Staphylococcus epidermidis (SE) and combinations of any two or more of these;
step (a) comprises at least one of mechanical lysis, chemical lysis and a combination of mechanical and chemical lysis;
the first capture probes are secured to a substrate during steps (b) and (c);
the substrate is substantially planar and the first capture probes are secured within a first detection region;
the substrate comprises at least one of plastic, metal, glass, nitrocellulose, organic material and a magnetic-bead based platform;
the magnetic-bead based platform comprises a Luminex MAGPIX™ system;
step (c) is carried out by a quantitative real-time PCR (qPCR) system;
step (c) comprises at least one of excitation and imaging of fluorescent-tagged detector probes; bioluminescence using luciferase-type enzymes; and amperometric current using an electrochemical sensor;
the clinical specimen is suspected of containing pathogens associated with at least one of a urinary tract infection and sepsis;
the specimen is a clinical specimen;
the clinical specimen comprises at least one of urine, blood, serum, plasma, saliva, tears, gastric fluids, digestive fluids, stool, mucus, sputum, sweat, earwax, oil, semen, vaginal fluid, glandular secretion, breast milk, synovial fluid, pleural fluid, lymph fluid, amniotic fluid, feces, cerebrospinal fluid, wounds, burns, tissue homogenates and an inoculum derived therefrom that is generated during conventional laboratory testing procedures;
steps (a) to (c) are conducted in sequence on the specimen without an intervening culturing or incubation step; and/or
the first oligonucleotide probe set further comprises a helper probe having sequences adapted to hybridize with a helper sequence on the first capture probe to help immobilize the first capture probe during steps (b) and (c).

In another of its aspects, the present invention relates to an oligonucleotide probe set for use in identifying a first target microbe in a specimen, wherein the oligonucleotide probe set comprises a capture probe and a detector probe, the capture probe and detector probe each being adapted to selectively hybridize to a first target sequence of rRNA molecules released from the first target microbe.

Preferred embodiments of this oligonucleotide probe set may include any one or a combination of any two or more of any of the following features:
the probe set further comprises a helper probe adapted to hybridize with a helper sequence of the capture probe to help immobilize the first capture probe;
the probe set is adapted to selectively bind to rRNA molecules released from a pre-determined microorganism, preferably a microorganism selected from the group consisting of Escherichia coli (EC), Klebsiella pneumoniae (KP), Proteus mirabilis (PM), Pseudomonas aeruginosa (PA), Staphylococcus saprophyticus (SS), Staphylococcus aureus (SA), Streptococcus agalactiae (GB), Enterococcus faecium (EM), Candida albicans (CA), Enterobacteriaceae (EB), Enterococci (EF), Candida species (CN), Citrobacter freundii (CF), Serratia marcescens (SM), Enterobacter cloacae (EL), Enterobacter hormaechei (EH), and Klebsiella oxytoca (KX), Klebsiella aerogenes (KA), Morganella morganii (MM), Acinetobacter baumanii (AB), Streptococcus pyogenes (SY), Streptococcus pneumoniae (SP), Streptococcus viridans (SV), Stenotrophomonas maltophilia (XM), Staphylococcus epidermidis (SE) and combinations of any two or more of these;
the oligonucleotide probe set is adapted to hybridize with rRNA molecules released substantially all eubacteria (EU) or similar microbes; and/or
the oligonucleotide probe set is adapted to hybridize 16S rRNA sequences.

In another of its aspects, the present invention relates to a probe panel disposed on a substrate, the probe panel comprising a plurality of detection regions, each detection region comprising an oligonucleotide probe set configured to selectively bind to RNA molecules released from a pre-determined microorganism, each detection region comprising a different oligonucleotide probe set.

Preferred embodiments of this probe panel may include any one or a combination of any two or more of any of the following features:
each oligonucleotide probe set comprises a first capture probe having an oligonucleotide adapted to hybridize with a first target sequence of the rRNA molecules of the pre-determined microorganism, and a first detector probe having a detectably labeled oligonucleotide adapted to hybridize with a second sequence of the rRNA molecules of the pre-determined microorganism;
the pre-determined microorganism is selected from the group consisting of: Escherichia coli (EC), Klebsiella pneumoniae (KP), Proteus mirabilis (PM), Pseudomonas aeruginosa (PA), Staphylococcus saprophyticus (S S), Staphylococcus aureus (SA), Streptococcus agalactiae (GB), Enterococcus faecium (EM), Candida albicans (CA), Enterobacteriaceae (EB), Enterococci (EF), Candida species (CN), Citrobacter freundii (CF), Serratia marcescens (SM), Enterobacter cloacae (EL), Enterobacter hormaechei (EH), and Klebsiella oxytoca (KX), Klebsiella aerogenes (KA), Morganella morganii (MM), Acinetobacter baumanii (AB), Streptococcus pyogenes (SY), Streptococcus pneumoniae (SP), Streptococcus viridans (SV), Stenotrophomonas maltophilia (XM), Staphylococcus epidermidis (SE) and combinations of any two or more of these;
the number of detection regions on the panel is between about 3 and 100, or between about 5 and 50, or between about 10 and 25;
the substrate comprises at least one of plastic, metal, glass, nitrocellulose, organic material and a magnetic-bead based platform.
the magnetic-bead based platform comprises a Luminex MAGPIX™ system.

In another of its aspects, the present invention relates to a system for identifying at least two microbes in a specimen, the system comprising:
(a) a probe panel disposed on a substrate, the probe panel comprising a plurality of detection regions, each detection region comprising an oligonucleotide probe set configured to selectively bind to RNA molecules released from a pre-determined target microbe, each detection region comprising a different oligonucleotide probe set; and
(b) a detection apparatus configured to detect hybridized complexes present in any detection regions of the probe panel.

Preferred embodiments of this system may include any one or a combination of any two or more of any of the following features:
the detection apparatus comprises a plurality of sensors, each sensor associated with a single detection region and configured to detect a signal from a hybridized complex when present in the single detection region;
the plurality of sensors are operable simultaneously;
the detection apparatus comprises a single sensor, moveable to be associated with each detection region and configured to detect a signal from a hybridized complex when present in a given detection region; and/or
wherein the specimen is selected from the group consisting of urine, blood, serum, plasma, saliva, tears, gastric fluids, digestive fluids, stool, mucus, sputum, sweat, earwax, oil, semen, vaginal fluid, glandular secretion, breast milk, synovial fluid, pleural fluid, lymph fluid, amniotic fluid, feces, cerebrospinal fluid, wounds, burns, tissue homogenates and an inoculum derived therefrom that is generated during conventional laboratory testing procedures.

Various apparatuses or processes will be described below to provide an example of an embodiment of each claimed invention. No embodiment described below limits any claimed invention and any claimed invention may cover processes or apparatuses that differ from those described below. The claimed inventions are not limited to apparatuses or processes having all of the features of any one apparatus or process described below or to features common to multiple or all of the apparatuses described below. It is possible that an apparatus or process described below is not an embodiment of any claimed invention. Any invention disclosed in an apparatus or process described below that is not claimed in this document may be the subject matter of another protective instrument, for example, a continuing patent application, and the applicants, inventors, or owners do not intend to abandon, disclaim, or dedicate to the public any such invention by its disclosure in this document.

The term “specimen” used herein refers to a material which is isolated from its natural environment, including but not limited to biological materials (see definition of “clinical specimen” below), food products, and fermented products.

The term “clinical specimen” used herein refers to samples of biological material, including but not limited to urine, blood, blood cultures (such as may be prepared when diagnosing sepsis), cultures of other biological material, serum, plasma, saliva, tears, gastric and/or digestive fluids, stool, mucus, sputum, sweat, earwax, oil, semen, vaginal fluid, glandular secretion, breast milk, synovial fluid, pleural fluid, lymph fluid, amniotic fluid, feces, cerebrospinal fluid, wounds, and tissue homogenates or an inoculum derived therefrom that is generated during conventional laboratory testing procedures. The clinical specimen may be collected and stored by any means, including in a sterile container.

A clinical specimen may be provided by or taken from any mammal, including but not limited to humans, dogs, cats, murines, simians, farm animals, sport animals, and companion animals.

The term “microbe” used herein refers to any species of microorganism, including but not limited to bacteria, fungi, and parasites.

The term “bacteria” used herein refers to any species of bacteria, including but not limited to Gram-negative and Gram-positive bacteria, and anaerobic bacteria.

The term “rRNA” used herein refers to the ribosomal ribonucleic acid of microbes present in the clinical specimen.

The term “positive control” used herein refers to a known concentration of a target molecule that is included in an assay to produce a known and expected effect. Examples of target molecules that can be used as positive controls would be known to the person skilled in the art, and include synthetic oligonucleotides that have the same sequence as the target rRNA sequence.

The term “negative control” used herein refers to a known treatment that is included in an assay that is not expected to have any effect. Examples of treatments that can be used as negative controls would be known to the person skilled the art, and include specimens that do not contain rRNA, including RNAse-treated samples.

The term “background” used herein refers to the result obtained from samples lacking rRNA, bacteria, or other microbes.

There is an urgent need for timely and accurate methods of microbial identification in specimens, including, for example clinical specimens. In certain circumstances, it is important to detect and identify microbial pathogens in a clinical specimen in the diagnosis and/or treatment of infectious disease. Accurately detecting and identifying the presence of microbial species or a combination thereof in the clinical specimen, may help determine an appropriate course of treatment. Information regarding the presence (or absence) of certain species of microbes in a given clinical specimen may also be of assistance to improve performance of antimicrobial susceptibility testing.

Often, the goal of such testing is to address potential drug resistance in common microbial pathogens, and to assure susceptibility of the microbial pathogens to drugs of choice for particular infections. Information obtained in a microbial identification screen may help clinicians prescribe the appropriate effective antibiotics or other treatment regimes. Timely detection and identification of the particular species of microbial pathogen in a clinical specimen, alone or in combination with antimicrobial susceptibility testing, may also assist clinicians in prescribing a targeted antibiotic or other treatment, to reduce the risk of over-prescription and evolution of drug-resistance microbes. Such methods of identification may also be used by public health officials to address the growing concern of drug-resistant infections and epidemics.

For example, a given clinical specimen may be obtained from a patient, whether it be a human or animal, who may require further medical treatment based on the results of the analysis of the clinical specimen. For example, urine specimens are often obtained from patients experiencing symptoms consistent with urinary tract infections (UTIs). In other examples, blood culture specimens may be prepared from blood samples obtained from patients experiencing symptoms consistent with sepsis.

However, conventional methods of identifying microbes in a clinical specimen, such as from a urine specimen, are relatively slow and cumbersome, requiring scarce time and resources. For example, a conventional method for collecting a clinical specimen and identifying a microbial pathogen may require a microbial culture with at least one growth phase or other amplification methods with long incubation periods of the target to be identified. Such methods may be relatively accurate but may tend to be relatively slow, taking several hours, days or weeks to provide useful results to the clinician. In a clinical environment, such time frames may be undesirable and may be considered too long a time period to withhold/delay treatment for the patient.

That is, while conventional techniques for identifying microbes in a clinical specimen may tend to produce generally accurate results, they may be considered too slow to be of practical assistance. This time delay can sometimes lead to treatments being implemented, such as a particular antibiotic being prescribed to a patient, before the results from a microbial identification screen are obtained. This can sometimes lead to the unnecessary prescription antibiotics and/or the prescription of a selected antibiotic that is less effective in treating a particular class of microbes than other available antibiotics.

In addition to the time required to perform the analysis, conventional techniques often require a skilled technician to set-up and run the bacteria cultures, as well as to interpret the results. The analysis may also require specialized and/or costly equipment. As such equipment and skilled technicians are relatively scarce resources, they are often located in centralized labs and/or hospital environments which are removed from common frontline care facilities, such as a physician's or veterinarian's office, walk-in clinics, and the like. This arrangement can further delay the processing and analysis of clinical specimens by several hours or days, as the specimens must be physically transported from the front-line environment to a centralized testing location and may then wait in a testing queue or backlog of samples awaiting analysis. This time-delay may reduce the accuracy of the ensuing clinical specimen analysis due to such factors as growth or death of any bacteria that may be present in the specimen.

There remains a need for relatively faster specimen analysis methods, and a need to be able to perform at least some of the analysis in situ in a front-line setting, such as in a physician's or veterinarian's office, instead of having to physically transport the clinical specimens to a centralized location. Similarly, it would be advantageous to provide a method in which a clinically meaningful test result (i.e. information that can help inform treatment decisions) can be provided to a care giver without requiring the individual skill and judgement of a skilled technician.

There also remains a need for a method of identifying microbes within a specimen that includes directly assaying the “raw” specimen, without requiring an intervening culturing step (or other material pre-processing step) in the identification testing process itself, which may delay the testing process and possibly contribute to inaccuracies as described herein. That is, the specimen to be examined using these methods may be an unprocessed biological sample from a patient (such as a urine sample) or may have undergone some degree of pre-processing and may be an inoculant or culture of a biological sample (such as a blood culture prepared as part of a sepsis diagnosis protocol) prior to the initiation of the identification process, but does not require any further culturing or processing as part of the rRNA-based identification process described herein.

To help overcome at least some of these deficiencies in conventional methods of clinical specimen analysis, the present inventors have developed a method in which it is possible to identify microbial species directly in a clinical specimen in situ, in a front-line setting, and in less time than conventional methods may allow for.

The present inventors have developed some species-specific oligonucleotide probe sets, each comprising a capture probe and a detector probe (which together form a probe pair), which are optimized to hybridize with target sequences in rRNA molecules within given microbes which may facilitate uniquely identifying a microbial species, microbial family or a microbial genus. Optionally, multiple types of differently-optimized probe sets may be coordinated such that they can each capture their target rRNA molecules without capturing (in a material manner) rRNA molecules that are the focus of different ones of the probe sets. This may allow multiple types of probe sets to be used simultaneously on a specimen containing a combination microbes to provide discrete detection/identification of different microbes based on the capture rRNA molecules by the different sets of probes.

Optionally, in addition to a capture probe and a detector probe, the probe sets may include a third, helper probe that may help improve, and may optionally optimize, the functioning of the capture and detector probes of the present invention. In some embodiments, the helper probes may hybridize with certain oligonucleotide helper sequences provided on a given capture probe to help immobilize the capture probes.

The present inventors have also developed methods of using said probes for rapid identification of microbial species in clinical specimens. The methods may be used, for example, in identifying common microbial species in clinical specimens obtained from patients suspected of having a UTI.

The present inventors have determined that rRNA is an ideal analyte in a method for microbial identification. rRNA is present in relatively high quantities (up to approximately 100,000 copies per cell) in clinically-relevant microbes. The number of rRNA copies is such that direct detection is feasible without an additional amplification step, which may be required in other conventional methods and which may be relatively time-consuming and labor intensive. Moreover, microbial rRNA contain unique signature sequences that are highly accessible to hybridization by oligonucleotide probes of the present invention, which may enable species-specific identification.

As further described herein, the inventors have developed particular oligonucleotide probes which rapidly hybridize with specific target rRNA sequences of clinically-relevant microbial species within a clinical specimen.

The present inventors have also optimized the probes of the present invention such that said probes may be used in combination such as in a panel comprising a plurality of probes, which may be used on a clinical specimen reliably. In contrast, some previously known methods and oligonucleotide probes may have been relatively disadvantageous as they were less target-specific or had the problem of causing interference with probes adapted to detect other species such that the previously known probes could not be reliably used in a single mixture.

The present inventors have also developed a probe panel comprising of a plurality of probe sets, preferably 13 probe sets, wherein each probe set is uniquely adapted to identify a particular family, genus or species of microbe, which permits rapid and reliable identification in a clinical specimen. In one embodiment, the probe panel of the present invention may be particularly useful for clinicians in determining treatment options for UTIs or sepsis, for example.

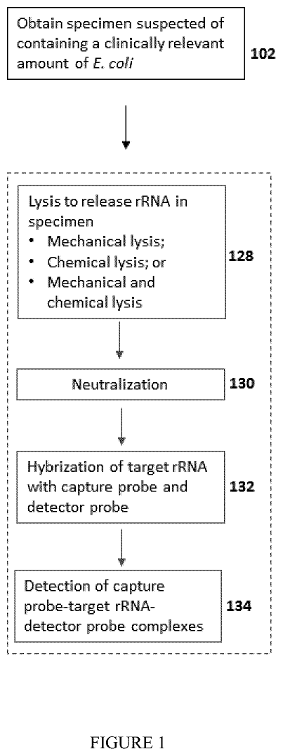

In accordance with one aspect of the teachings described herein, a method of identifying microbial species in a clinical specimen is described. FIG. 1 is a flowchart illustrating one embodiment of this method.

Referring to FIG. 1, one example of a method 100 of identifying microbial species in a clinical specimen includes a first step 102 of obtaining a clinical specimen. In most embodiments of the method, the clinical specimen is believed to contain at least one microbial species in a clinically relevant amount, and may be suspected of containing two or more microbial species in a clinically relevant amount.

Once the specimen suspected of containing a clinically relevant amount of one or more microbial species is obtained, the specimen is processed to detect the presence of any target microbial species.

Detecting the presence of microbial species may be done using a suitable method such as the method described herein. One example of a suitable method may include the steps of: 1) lysis to release rRNA 128; 2) Neutralization 130; 3) Hybridization of target rRNA with a probe set 132; and 4) Detection of hybridized complexes 134, comprising capture probe—target rRNA—detector probe.

In the illustrated example, a MagPix (Luminex) magnetic bead assay is used to identify the microbial species in fresh urine specimens from a patient with UTI.

### Lysis (Step 128)

Optionally, the lysing step 128 may include at least one of chemical lysing, mechanical lysing, and/or a combination thereof. In a preferred embodiment, lysis 128 may include both chemical and mechanical lysing operations. In a more preferred embodiment, the chemical and mechanical lysing operations may be performed simultaneously. Alternatively, the chemical and mechanical lysing operations may be performed at different times. One example of a suitable lysing method and apparatus is described in the U.S. provisional patent No. 62/541,418 (“RiboLyse”), which is incorporated herein by reference.

Lysis by the method described herein may ideally release rRNA from the microbial cells which may be present in the clinical specimen thereby exposing said rRNA to oligonucleotide probes. In one embodiment of the present invention, the lysing step 128 may be completed in about 5 minutes.

### Neutralization (Step 130)

The goal of the neutralization step is to get the lysate to a pH between about 6 and about, preferably about between 6.5 and about 7.5, most preferably, about 7. The neutralization step 130 can be performed using any known or unknown method.

In the illustrated example, samples are lysed with one-half sample volume of 1M NaOH. This lysate is then neutralized with an equal volume (1.5× sample volume) of 1M sodium-potassium phosphate buffer, pH 6.4.

### Hybridization (Step 132)

The oligonucleotide probe sets of the present invention are adapted to hybridize with a specific target rRNA sequence uniquely appearing in a target microbial family, genus or species, as the case may be. Preferably, a distinct signal pattern may be provided for each type of target microbe that may be present in a clinical specimen to facilitate multiplexed detection. That is, the detector probe used in the different types of probe sets can be configured so that its presence can be distinguished from the presence of the detector probes in other ones of the probe sets. This may be done based on the nature in which the detector probes are detected, by spatially segregating the different types of probe sets into different detection regions or on different, separately observable substrates or other suitable techniques.

As used herein, the term hybridized complexes can be used to refer to the combination of a probe set and its captured rRNA molecule. That is, the combination of one capture probe and one detector probe hybridized to a common rRNA molecule.

Preferably, a species-specific signal can be provided for each type of target microbe that may be present in the clinical specimen. By using a species-specific signal, the signal of rRNA from different types of microbe in mixed specimens may be individually observed together at substantially the same time and/or only signals from the desired, targeted microbes may be observed. This may help facilitate the identification of two or more different target microbes within a single clinical specimen, and may allow the presence of two or more target microbes to be detected generally simultaneously.

This may be advantageous when analyzing certain types of clinical specimens, such as urine specimens, which may tend to include a variety of different microbes in generally unknown quantities at the beginning of the analysis process. By using species-specific signal probes, the methods described herein could be used to independently detect rRNA from two or more specific microbial species in the clinical specimen.

### Detection (Step 134)

A variety of platforms can be used for detection 134, including but not limited to excitation and imaging of fluorescent-tagged detector probes, bioluminescence using luciferase-type enzymes, amperometric current using an electrochemical sensor, and using a quantitative real-time PCR (qPCR) system. In the illustrated example, fluorescent-tagged detector probes are used for detection.

Optionally, the specimen may be processed after the hybridization steps to help remove unattached reagents (i.e. capture probes, detector probes) and other material that need not be measured or detection during the detection phase. For example, as part of the detection process the specimen may be washed after hybridization is finished so was to wash away detector probe that are not part of a hybridized complex so that the remaining detector probe present in the sample substantially correspond to the presence of their corresponding rRNA molecules. When the unattached detector probes are removed, the presence of the remaining detector probes that are part of hybridized complexes, can then be used to indicate the associated microbe that is the target of that given type of probe set is also present in the specimen.

During detection 134, at least one positive control and at least one negative control can be included. In the illustrated example using the probe panel 200, a synthetic oligonucleotide with the same sequence as the target rRNA is included as a positive control and a sample without rRNA or bacteria is included as a negative control. Synthetic oligonucleotide target sequences for each target family, genus or species, as used in an embodiment of the present invention are provided in Tables 1 and 2 (microbe identification abbreviations corresponding to Tables 1 and 2 are provided in Tables 3 and 4, respectively). Table 2 in particular provides synthetic oligonucleotide target sequences which may be targeted to gram negative bacterial pathogens.

TABLE 1
Synthetic Target Oligonucleotide Sequences for Detection of Microbial Species
or Genus

| Microbe (length) | Sequence (5′-3′) |
|---|---|
| EU GN (37m) | CTGGGGTGAAGTCGTAACAAGGTAACCGTAGGGGAAC (SEQ ID NO. 72) |
| EU GP (37m) | CTGGGGTGAAGTCGTAACAAGGTAGCCGTATCGGAAG (SEQ ID NO. 73) |
| EC (30m) | TCAGCGGGGAGGAAGGGAGTAAAGTTAATA (SEQ ID NO. 74) |
| SG 32m | AGAAGAACGTTGGTAGGAGTGGAAAATCTACC (SEQ ID NO. 75) |
| EM 31m | TTAGAGAAGAACAAGGATGAGAGTAACTGTT (SEQ ID NO. 76) |
| PA 25m | TAAGTTGGGAGGAAGGGCAGTAAGT (SEQ ID NO. 77) |
| SA 32m | CGGTACTCGTTAAGGCTGAGCTGTGATGGGGA (SEQ ID NO. 78) |
| SS 33m | GGATAACATTTGGAACCGCATGGTTCTAAAGTG (SEQ ID NO. 79) |
| EB 46m | ATACAAAGAGAAGCGACCTCGCGAGAGCAAGCGGACCTCATAAAGT (SEQ ID |
| | NO. 80) |
| PM 26m | TCAGCGGGGAGGAAGGTGATAAGGTT (SEQ ID NO. 81) |
| KP 26m | AGCTGAGACCAGTCGAAGATACCAGC (SEQ ID NO. 82) |
| CN 36m | CGAGTTGTTTGGGAATGCAGCTCTAAGTGGGTGATG (SEQ ID NO. 83) |
| EF 40m | AAAGGCGCTTTCGGGTGTCGCTGATGGATGGACCCGCGGT (SEQ ID NO. 84) |

TABLE 2
Synthetic Target Oligonucleotide Sequences for Detection of Microbial Species,
or Family Targeted to Gram negative bacterial pathogens

| Microbe (length) | Sequence (5′-3′) |
|---|---|
| EU GN (37m) | CTGGGGTGAAGTCGTAACAAGGTAACCGTAGGGGAAC (SEQ ID NO. 85) |
| EC (30m) | TCAGCGGGGAGGAAGGGAGTAAAGTTAATA (SEQ ID NO. 86) |
| KE (23m) | TCGARCGGTAGCACAGAGAGCTT (SEQ ID NO. 87) |
| PA (25m) | taagttgggaggaagggcagtaagt (SEQ ID NO. 88) |
| EHa 32m | TGCAAGTCGAACGGTAACAGGAAGCAGCTTGC (SEQ ID NO. 89) |
| EHb 34m | TGCAAGTCGAACGGTAACAGGTAAGCAGCTTGCT (SEQ ID NO. 90) |
| ELa 32m | CGGTACCTTTTAACGCTGAGGTGTGATGACGA (SEQ ID NO. 91) |
| ELb 33m | CGGTACCTTHTTAACGCTGAGGTGTGATGACGA (SEQ ID NO. 92) |
| EB 46m | ATACAAAGAGAAGCGACCTCGCGAGAGCAAGCGGACCTCATAAAGT (SEQ ID |
| | NO. 93) |
| PM 26m | TCAGCGGGGAGGAAGGTGATAAGGTT (SEQ ID NO. 94) |
| KP 26m | AGCTGAGACCAGTCGAAGATACCAGC (SEQ ID NO. 95) |
| SM 29m | CAGCGAGGAGGAAGGTGGTGAACTTAATA (SEQ ID NO. 96) |
| CF 28m | TTGCTCCTTGGGTGACGAGTGGCGGACG (SEQ ID NO. 97) |
| KX 33m | CGGAACGTCACTAACGCTGAGGTGTGATGACGA (SEQ ID NO. 98) |

### Oligonucleotide Probes

The oligonucleotide probes used in the microbial identification method 100 may be provided as described herein. For each target species, genus or family, the inventors have developed associated oligonucleotide probe sets comprising a capture probe and a detector probe. In some embodiments, the probe set further comprises a helper probe. Each probe may preferably comprise a sequence of fewer than 60 nucleotides and more preferably, between about 10 and about 50 nucleotides.

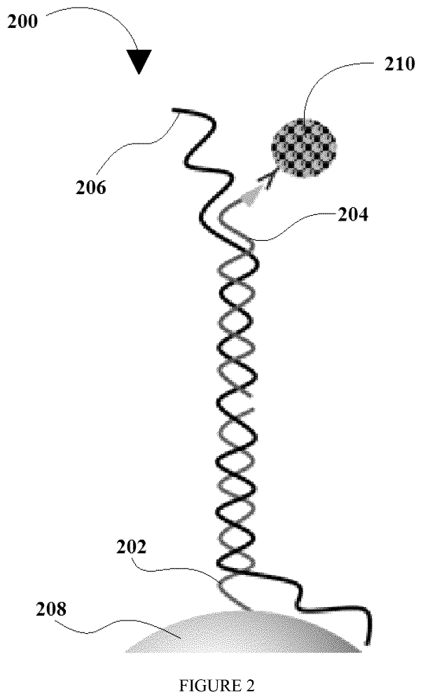

Referring to FIG. 2, probe set 200 is provided comprising capture probe 202 and detector probe 204. Capture probes and detector probes of the present invention are typically developed to hybridize specifically to a target capture region and a target detector region, respectively, of a target microbial rRNA 206.

The capture probe 202 comprises nucleotide bases which are complementary to the nucleic acid sequence in the target capture region of the target rRNA while a detector probe 204 comprises nucleotide bases which are complementary to the nucleic acid sequence in the target detector region. In some embodiments of the present invention, capture probes 202 may further include a helper sequence of nucleic acid base pairs which is adapted to hybridize with a helper probe.

Capture probe 202 further includes an anchoring means which anchors the capture probe to a solid substrate 208. In a preferred embodiment the substrate may be any type of generally solid material to which the capture probe 202 can be bonded, and may include metal, glass, plastic, organic material nitrocellulose, or other suitable substrate materials may be employed. The substrate may be configured as a generally flat or planar substrate that is suitable for holding a plurality of capture probes 202 in a generally planar array or matrix arrangement. Alternatively, the substrate may be configured as a bead or other three-dimensional object to which the capture probe 202 can be attached.

When capture probe 202 hybridizes with a capture region of a target rRNA, the target rRNA may thereby become anchored to the solid substrate 208 and facilitate detection.

An assay comprising an array of capture probes may be provided in embodiments utilizing a plurality of probes. Preferably, the capture probe array may be arranged in a regular pattern such that a detection means may readily detect hybridized probes based on recognition of a predetermined pattern.

Preferably, the detector probe 204 of the present invention can be detectably labelled using a suitable detection portion that is detectable using a detection machine, such as such as a fluorescent camera, for example, a Luminex MAGPIX™ system or equivalent thereof. For example, the detection portion may include a quantum dot nanocrystal 210 having pre-determined characteristics that are observable to a corresponding detection machine. In the preferred embodiment, the detection portion of the detector probe 204 is functionalized with biotin for interaction with streptavidin conjugated to a signaling molecule as shown schematically on FIG. 2. The detector probes are adapted to hybridize with a detector region of a target rRNA, wherein the hybridization enables detection by activating the signaling molecule.

The inventors have also discovered that there is a linear correlation between the assay signal obtained from hybridized detector probes and the target rRNA concentration. The method and probes described herein may also be used for other purposes such as estimating the concentration of a target microbe in the clinical specimen, as described in U.S. Provisional Application No. 62/671,380, filed May 14, 2018, the contents of which is incorporated herein by reference.

In some embodiments, a helper probe is provided to facilitate the hybridization of capture probes and detector probes. The present inventors have discovered that certain oligonucleotide probes may be optimized to function more efficiently by adding an additional probe hybridization step using a helper probe. The helper probe of the present invention may comprise two distinct sequences: a first sequence adapted to hybridize with a helper sequence of an associated capture probe and a second sequence adapted to hybridize with an anchoring or detection means such as a Luminex bead, wherein the hybridization of the helper probe and helper sequence may take place before a capture probe hybridizes with a target rRNA sequence and before the probe set is activated for detection by a detection means. In another embodiment, the helper probe may comprise a sequence adapted to hybridize with a helper sequence of an associated capture and an immobilization moiety. Helper probes of the present invention may assist in overcoming the problem of probe interference, reliability, and specificity.

The nucleotide sequences of representative capture probes, detector probes, and helper probes of the present invention are provided in Tables 3 and 4. In Tables 3 and 4, sequences of the capture probes which are used to hybridize with Luminex bead models provided in the example embodiment are denoted with square brackets. Sequences of the example capture probe and helper probes which are adapted to hybridize with each other (e.g. helper sequences) are denoted with parentheses (round brackets). In Table 3, sequences of capture probes, detector probes, and helper probes are provided which are configured to target common uropathogens. In Table 4, sequences of capture probes, detector probes, and helper probes are provided which are configured to target Gram negative bacterial pathogens.

TABLE 3
Sequences of capture probes, detector probes, and helper probes for hybridization
with rRNA of microbial species, genus or family in Uropathogens

| | | |
|---|---|---|
| Probe Set | Position* | Sequence (5′-3′) |

Gram-negative biased Eubacterial (Universal) (EU GN)

| Luminex Bead Number | | MTAG-A012 |
|---|---|---|
| Capture | 1484 | GTTACGACTTCACCCCAG [CATAATCAATTTCAACTTTCTACT] |
| | | (SEQ ID NO. 1) |
| Detector | 1502 | Biotin-GTTCCCCTACGGTTACCTT (SEQ ID NO. 2) |

Gram-positive biased Eubacteria (Universal) (EU GP)

| Luminex Bead Number | MTAG-A012 |
|---|---|
| Capture | GTTACGACTTCACCCCAG [CATAATCAATTTCAACTTTCTACT] |
| | (SEQ ID NO. 3) |
| Detector | Biotin-CTTCCGATACGGCTACCT (SEQ ID NO. 4) |

Escherichia coli (EC)

| Luminex Bead Number | | MTAG-A013 |
|---|---|---|
| Helper | | (TCATAATTCTACATCTATCACTTA) [CAAATACATAATCTTACATTCACT] |
| | | (SEQ ID NO. 5) |
| Capture | 454 | (TAAGTGATAGATGTAGAATTATGA) TATTAACTTTACTCC |
| | | (SEQ ID NO. 6) |
| Detector | 439 | CTTCCTCCCCGCTGA-Biotin (SEQ ID NO. 7) |

Enterobacteriaceae (EB)

| Luminex Bead Number | | MTAG-A025 |
|---|---|---|
| Helper | | (TCATAATTCTACATCTATCACTTA) [CTTTCTTAATACATTACAACATAC] |
| | | (SEQ ID NO. 8) |
| Capture | 1269 | (TAAGTGATAGATGTAGAATTATGA) ACTTTATGAGGTCCGCTTGCTCT |
| | | (SEQ ID NO. 9) |
| Detector | 1246 | CGCGAGGTCGCTTCTCTTTGTAT-Biotin (SEQ ID NO. 10) |

Klebsiella pneumoniae (KP)

| Luminex Bead Number | | MTAG-A033 |
|---|---|---|
| Helper | | (TCATAATTCTACATCTATCACTTA) [ACTACTTATTCTCAAACTCTAATA] |
| | | (SEQ ID NO. 11) |
| Capture | 1748 (23S) | (TAAGTGATAGATGTAGAATTATGA) GCTGGTATCTTCG |
| | | (SEQ ID NO. 12) |
| Detector | 1735 (23S) | ACTGGTCTCAGCT-Biotin (SEQ ID NO. 13) |

Proteus mirabilis (PM)

| Luminex Bead Number | | MTAG-A029 |
|---|---|---|
| Helper | | (TCATAATTCTACATCTATTACATC) [TACTACTTCTATAACTCACTTAAA] |
| | | (SEQ ID NO. 14) |
| Capture | 453 | (GATGTAATAGATGTAGAATTATGA) AACCTTATCAC (SEQ ID NO. 15) |
| Detector | 438 | CTTCCTCCCCGCTGA-Biotin (SEQ ID NO. 16) |

Pseudomonas aeruginosa (PA)

| Luminex Bead Number | | MTAG-A018 |
|---|---|---|
| Helper | | (TCATAATTCTACATCTATCACTTA) [ACACTTATCTTTCAATTCAATTAC] |
| | | (SEQ ID NO. 17) |
| Capture | 447 | (TAAGTGATAGATGTAGAATTATGA) ACTTACTGCCC (SEQ ID NO. 18) |
| Detector | 433 | TTCCTCCCAACTTA-Biotin (SEQ ID NO. 19) |

Enterococcus genus (EF)

| Luminex Bead Number | | MTAG-A037 |
|---|---|---|
| Helper | | (TCATAATTCTACATCTATCACTTA) [TACAACATCTCATTAACATATACA] |
| | | (SEQ ID NO. 20) |
| Capture | 186 | (TAAGTGATAGATGTAGAATTATGA) ACCGCGGGTCCATCCATCAG |
| | | (SEQ ID NO. 21) |
| Detector | 186 | CGACACCCGAAAGCGCCTTT-Biotin (SEQ ID NO. 22) |

Enterococcus faecium (EM)

| Luminex Bead Number | | MTAG-A015 |
|---|---|---|
| Helper | | (TCATAATTCTACATCTATCACTTA) [TACTTCTTTACTACAATTTACAAC] |
| | | (SEQ ID NO. 23) |
| Capture | 422 | (TAAGTGATAGATGTAGAATTATGA) AACAGTTACTCTCATC |
| | | (SEQ ID NO. 24) |
| Detector | 407 | CTTGTTCTTCTCTAA-Biotin (SEQ ID NO. 25) |

Streptococcus agalactiae (SG)

| Luminex Bead Number | MTAG-A014 |
|---|---|
| Capture | TACCAACGTTCTTCT [AATTTCTTCTCTTTCTTTCACAAT] |
| | (SEQ ID NO. 26) |
| Detector | Biotin-GGTAGATTTTCCACTCC (SEQ ID NO. 27) |

Staphylococcus aureus (SA)

| Luminex Bead Number | MTAG-A020 |
|---|---|
| Helper | (TCATAATTCTACATCTATCACTTA) [CTTTCTCATACTTTCAACTAATTT] |
| | (SEQ ID NO. 28) |
| Capture | (TAAGTGATAGATGTAGAATTATGA) TCCCCATCACAGCTCA |
| | (SEQ ID NO. 29) |
| Detector | GCCTTAACGAGTACCG-Biotin (SEQ ID NO. 30) |

Staphylococcus saprophyticus (SS)

| Luminex Bead Number | MTAG-A022 |
|---|---|
| Capture | TTCCAAATGTTATCC [CAAACAAACATTCAAATATCAATC] |
| | (SEQ ID NO. 31) |
| Detector | Biotin-CACTTTAGAACCATGCGG (SEQ ID NO. 32) |

Candida genus (CN)

| Luminex Bead Number | MTAG-A035 |
|---|---|
| Capture | GCATTCCCAAACAACTCG [CATCTTCATATCAATTCTCTTATT] |
| | (SEQ ID NO. 33) |
| Detector | Biotin-CATCACCCACTTAGAGCT (SEQ ID NO. 34) |

Candida albicans (CA)

| Luminex Bead Number | |
|---|---|
| Helper | (TTCTACATCACTTACATTTAACAT) ACTACTTATTCTCAAACTCTAATA |
| | (SEQ ID NO. 35) |
| Capture | (ATGTTAAATGTAAGTGATGTAGAA) CCTTCTTCAAATTACAACT |
| | (SEQ ID NO. 36) |
| Detector | CGGACGCCAAAGACGCCA-Biotin (SEQ ID NO. 37) |

*Denotes hybridization position of the 5′ nucleotide in alignment with the target rRNA molecule.

TABLE 4
Sequences of capture probes, detector probes, and helper probes for hybridization
with rRNA of microbial species, genus or family targeted to Gram negative bacterial
pathogens

| | | |
|---|---|---|
| Probe Set | Position* | Sequence (5′-3′) |

Gram-negative biased Eubacterial (Universal) (EU GN)

| Luminex Bead Number | | MTAG-A012 |
|---|---|---|
| Capture | 1484 | GTTACGACTTCACCCCAG [CATAATCAATTTCAACTTTCTACT] |
| | | (SEQ ID NO. 38) |
| Detector | 1502 | Biotin-GTTCCCCTACGGTTACCTT (SEQ ID NO. 39) |

Escherichia coli (EC)

| Luminex Bead Number | | MTAG-A013 |
|---|---|---|
| Helper | | (TCATAATTCTACATCTATCACTTA) [CAAATACATAATCTTACATTCACT] |
| | | (SEQ ID NO. 40) |
| Capture | 454 | (TAAGTGATAGATGTAGAATTATGA) TATTAACTTTACTCC |
| | | (SEQ ID NO. 41) |
| Detector | 439 | CTTCCTCCCCGCTGA-Biotin (SEQ ID NO. 42) |

Enterobacteriaceae (EB)

| Luminex Bead Number | | MTAG-A025 |
|---|---|---|
| Helper | | (TCATAATTCTACATCTATCACTTA) [CTTTCTTAATACATTACAACATAC] |
| | | (SEQ ID NO. 43) |
| Capture | 1269 | (TAAGTGATAGATGTAGAATTATGA) ACTTTATGAGGTCCGCTTGCTCT |
| | | (SEQ ID NO. 44) |
| Detector | 1246 | CGCGAGGTCGCTTCTCTTTGTAT-Biotin (SEQ ID NO. 45) |

Klebsiella pneumoniae (KP)

| Luminex Bead Number | | MTAG-A033 |
|---|---|---|
| Helper | | (TCATAATTCTACATCTATCACTTA) [ACTACTTATTCTCAAACTCTAATA] |
| | | (SEQ ID NO. 46) |
| Capture | 1748 (23S) | (TAAGTGATAGATGTAGAATTATGA) GCTGGTATCTTCG |
| | | (SEQ ID NO. 47) |
| Detector | 1735 (23S) | ACTGGTCTCAGCT-Biotin (SEQ ID NO. 48) |

Proteus mirabilis (PM)

| Luminex Bead Number | | MTAG-A029 |
|---|---|---|
| Helper | | (TCATAATTCTACATCTATTACATC) [TACTACTTCTATAACTCACTTAAA] |
| | | (SEQ ID NO. 49) |
| Capture | 453 | (GATGTAATAGATGTAGAATTATGA) AACCTTATCAC |
| | | (SEQ ID NO. 50) |
| Detector | 438 | CTTCCTCCCCGCTGA-Biotin (SEQ ID NO. 51) |

Pseudomonas aeruginosa (PA)

| Luminex Bead Number | | MTAG-A018 |
|---|---|---|
| Helper | | (TCATAATTCTACATCTATCACTTA) [ACACTTATCTTTCAATTCAATTAC] |
| | | (SEQ ID NO. 52) |
| Capture | 447 | (TAAGTGATAGATGTAGAATTATGA) ACTTACTGCCC |
| | | (SEQ ID NO. 53) |
| Detector | 433 | TTCCTCCCAACTTA-Biotin (SEQ ID NO. 54) |

Klebsiella and Enterobacter (KE)

| Luminex Bead Number | MTAG-A014 |
|---|---|
| Helper | (TCATAATTCTACATCTATCACTTA) [AATTTCTTCTCTTTCTTTCACAAT] |
| | (SEQ ID NO. 55) |
| Capture | (TAAGTGATAGATGTAGAATTATGA) AAGCTCTCTGT |
| | (SEQ ID NO. 56) |
| Detector | GCTACCGYTCGA-Biotin (SEQ ID NO. 57) |

Enterobacter cloacae (EL)

| Luminex Bead Number | MTAG-A022 |
|---|---|
| Helper | (TCATAATTCTACATCTATCACTTA) [CAAACAAACATTCAAATATCAATC] |
| | (SEQ ID NO. 58) |
| Capture | (TAAGTGATAGATGTAGAATTATGA) TCGTCATCACACCTC |
| | (SEQ ID NO. 59) |
| Detector a | AGCGTTAAAAGGTACCG-Biotin (SEQ ID NO. 60) |
| Detector b | AGCGTTAADAAGGTACCG-Biotin (SEQ ID NO. 61) |

Enterobacter hormaechei (EH)

| Luminex Bead Number | MTAG-A020 |
|---|---|
| Helper | (TCATAATTCTACATCTATCACTTA) [CTTTCTCATACTTTCAACTAATTT] |
| | (SEQ ID NO. 62) |
| Capture a | (TAAGTGATAGATGTAGAATTATGA) GCAAGCTGCTTCCTGT- |
| | (SEQ ID NO. 63); |
| Capture b | (TAAGTGATAGATGTAGAATTATGA) AGCAAGCTGCTTACCTGT |
| | (SEQ ID NO. 64) |
| Detector | TACCGTTCGACTTGCA-Biotin (SEQ ID NO. 65) |

Serratia marcescens (SM)

| Luminex Bead Number | MTAG-A035 |
|---|---|
| Capture | CTTCCTCCTCGCTG [CATCTTCATATCAATTCTCTTATT] |
| | (SEQ ID NO. 66) |
| Detector | Biotin-TATTAAGTTCACCAC (SEQ ID NO. 67) |

Citrobacter freundii (CF)

| Luminex Bead Number | MTAG-A037 |
|---|---|
| Capture | CACCCAAGGAGCAA [TACAACATCTCATTAACATATACA] |
| | (SEQ ID NO. 68) |
| Detector | Biotin-CGTCCGCCACTCGT (SEQ ID NO. 69) |

Klebsiella oxytoca (KX)

| Luminex Bead Number | MTAG-A015 |
|---|---|
| Capture | AGCGTTAGTGACGTTCCG [TACTTCTTTACTACAATTTACAAC] |
| | (SEQ ID NO. 70) |
| Detector | Biotin-TGCTCATCACACCTC (SEQ ID NO. 71) |

*Denotes hybridization position of the 5′ nucleotide in alignment with the target rRNA molecule.

Using the methods and probes described herein, the presence or absence of a target species or genus of microbe may be determined by detecting the presence or absence of an activated signal from the corresponding hybridized detector probes.

### Probe Panels

Optionally, suitable ones of the oligonucleotide probe sets described herein can be grouped together, preferably in a complimentary manner, to provide a group or panel of probe sets, each of which is configured to target a different microbe. Pre-arranged probe panels of this nature may be provided to users to help facilitate the simultaneous detection of different types of microbes within a common specimen. For example, a specimen to be tested may contain an unknown quantity of microbes, and may include two or more different types of microbes. To help determine which microbes, if any, are present in the specimen, the specimen may be exposed to a pre-arranged probe panel that includes 2, 3, 4, 5, 6, 7, 8, 9, 10, 11, 12, 13, 14, 15 or more types of differently optimized/configured probe sets (preferably a plurality of each different type of probe set are present). During the assay, each probe set may capture its specific target microbe if present in the sample. At the conclusion of the assay the panel may be observed (using any suitable detection machine or in other manner) to determine which type of probe sets have captured target molecules (if any) and which have not. A positive presence of captured rRNA molecules in a given type of probe pairs can indicate that the specimen includes microbes of the associated type. That is, if the EC, KP and PM probe sets (as described herein) in a given panel capture their target rRNA molecules then it can be inferred that the specimen includes E. coli, K. pneumoniae and P. mirabilis microbes, but may not include other microbes that were also be tested for.

The selection of which types of probe sets are to be included together on a given probe panel may be motivated by a number of factors, including attempting to reduce the likelihood that two different probe sets on the panel are configured to target the same sequence of a target rRNA molecule (to help prevent cross-counting), and/or based on the identity of the microbes or mixture of microbes that a given specimen may be like to include. For example, a panel may be configured for use with a urine sample may be configured to include probe sets that are configured to target the types of microbes that a commonly found in urine samples from patients that have a UTI. While not detecting every possible microbe in the sample, such a panel may be a useful tool to quickly screen for the most likely microbes. Similarly, a panel may be configured for use with a blood culture specimen may be configured to include probe sets that are configured to target the types of microbes that a commonly found in blood cultures obtained from patients with sepsis, and may provide a relatively useful tool to screen a patient's blood culture for the most likely sepsis-causing pathogens. The probe sets used on the urine testing panel may be entirely different than the probe sets used on a blood culture testing panel, or the panels may include at least some of the same probe sets (e.g. if a given microbe happens to be common in both UTI and sepsis patients).

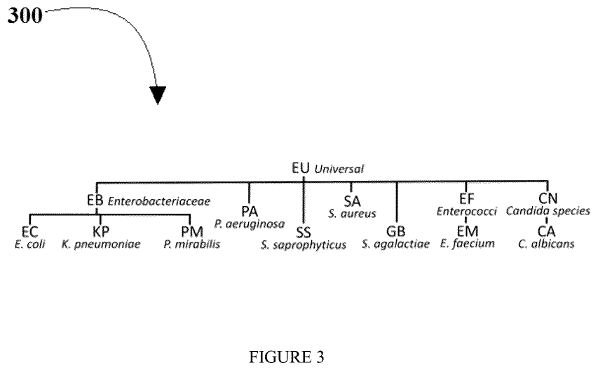

The oligonucleotide probes described herein may be used together as a probe panel, comprising a single multiplexed assay with a plurality of predetermined probe sets. In one preferred embodiment of the present invention as shown schematically in FIG. 3, probe panel 300 comprises 13 probe sets which have been selected to optimize microbial identification for use in UTI management and treatment. The plurality of different probe sets used to form this probe panel 300 may be arranged physically in an array on a carrier substrate with like types of probe pairs grouped together in pre-determined regions, or other suitable ways.

Each of the 13 probe sets within probe panel 300 are adapted to target a specific genus or species of microbe comprising: Escherichia coli (EC), Klebsiella pneumoniae (KP), Proteus mirabilis (PM), Pseudomonas aeruginosa (PA), Staphylococcus saprophyticus (SS), Staphylococcus aureus (SA), Streptococcus agalactiae (GB), Enterococcus faecium (EM), and Candida albicans (CA). In addition, probe panel 300 includes genus-specific and family-specific probes adapted to target a specific genus or family (which may include a plurality of species including those for which probe panel 300 does not include a species-specific probe set): Enterobacteriaceae (EB), Enterococci (EF), Candida species (CN). Further, probe panel 300 includes a universal probe (EU) which is adapted to target substantially all microbial 16S rRNA sequences of eubacteria or similar microbes.

The present inventors have selected the target microbes and developed corresponding probe sets through a rational basis in balancing the needs for clinical relevance, reliability, and cost, in particular for testing of clinical specimens (urine) relevant for UTIs.

The selection of the 13 probe sets in probe panel 300 may permit rapid and reliable microbial identification for use in UTI management and treatment, while balancing the need for sufficient relevant information to be provided to a clinician. In contrast, conventional assays may include many dozens or hundreds of probes, which increases cost and complexity yet the additional information may not be sufficiently relevant to outweigh their disadvantages.

The design of probe panel 300 also permits hierarchical identification of microbial species, such as for example the identification of an Enterococci which is not Enterococcus faecium. The addition of genus-specific probes and a universal probe also permits verification of results by introducing a positive control, thereby improving reliability of results.

Certain species-specific probes have been selected particularly for UTI management in a variety of settings. For instance, while a variety of Enterobacteriaceae may occur in urine specimens, those that require species-specific identification for management purposes are Escherichia coli, Klebsiella pneumoniae, and Proteus mirabilis. Among Enterococci, E. faecium merits specific identification because this organism tends to be resistant to antibiotics. Pseudomonas aeruginosa may be particularly resistant to antibiotics and merits specific identification. Although group B streptococcus (S. agalactiae) is considered a contaminant in urine specimens from most patients, a probe set for this organism is considered valuable in the obstetrics setting.

Further rationale for selection of the 13 microbial targets include:
a. EU: Eubacterial/universal—Inclusion of a universal probe pair enables detection of any organisms not detected by any of the other more specific probe pairs.
b. EC: Escherichia coli—Most common urine isolate, identification indicates treatment with nitrofurantoin or fosfomycin would be successful.
c. KP: Klebsiella pneumoniae—2^{nd} most common urine isolate, identification indicates intrinsic resistance to ampicillin.
d. PM: Proteus mirabilis—3^{rd} most common urine isolate, identification indicates intrinsic resistance to nitrofurantoin and risk of urinary tract stone formation.
e. EB: Enterobacteriaceae—Detection of members of the Enterobacteriaceae family that are not E. coli, K. pneumoniae or P. mirabilis can be handled as a group for treatment and management purposes.
f. PA: Pseudomonas aeruginosa—4^{th} most common Gram-negative urine isolate, identification indicates intrinsic resistance to nitrofurantoin and specific beta-lactam antibiotics.
g. EF/EM: Enterococcus spp./Enterococcus faecium—The most common Gram-positive urine isolate, frequently treatable with ampicillin except in the case of E. faecium.
h. SG: Streptococcus agalactiae—Frequently a contaminant of urine specimens, important to include in special circumstances such as pregnancy because carriage puts risk of newborn to meningitis.
i. SA: Staphylococcus aureus—Identification of S. aureus in the urine may indicate complicated or descending urinary tract infection, the latter may suggest systemic infection.
j. SS: Staphylococcus saprophyticus—A well-known Gram-positive uropathogen, which requires treatment.
k. CN/CA: Candida spp./Candida albicans—Frequently a contaminant of urine specimens, important to include in immunosuppressed patients who may be at risk of systemic infection. Identification of C. albicans indicates susceptibility to fluconazole.

In a preferred embodiment, capture probes as described above corresponding to each of the 13 targets of probe panel 300 may be arranged in a predetermined regular grid pattern on a substrate, with each predetermined position corresponding to the capture probe for a specific target microbe. A lysed clinical specimen may be contacted with the capture probe array and a mixture of detector probes corresponding to each of the 13 targets of probe panel 300. Signals from activated detector probes (those which have hybridized with target rRNA sequences) may be detected by detection means, such as a fluorescent camera system. The pattern of signal detection arranged in a regular grid pattern corresponding to the placement of the capture probes may be used for detection, thereby producing a unique output signal pattern corresponding to each target microbe.

Referring to FIG. 6, another example of a probe panel 400 is illustrated. This probe panel comprises 12 probe sets which have been selected to optimize microbial identification for use in the management and treatment of blood sepsis. The plurality of different probe sets used to form this probe panel 400 may be arranged physically in an array on a carrier substrate with like types of probe pairs grouped together in pre-determined regions, or other suitable ways.

Each of the 12 probe sets within probe panel 400 are adapted to target a specific genus or species of microbe comprising: Pseudomonas aeruginosa (PA), Enterobacteriaceae (EB), Citrobacter freundii (CF), Serratia marcescens (SM), Proteus mirabilis (PM), Escherichia coli (EC), Enterobacter cloacae (EL), Enterobacter hormaechei (EH), Klebsiella pneumoniae (KP) and Klebsiella oxytoca (KX). In addition, probe panel 400 includes genus-specific and family-specific probes adapted to target a specific genus or family (which may include a plurality of species including those for which probe panel 400 does not include a species-specific probe set): Enterobacteriaceae (EB) and Klebsiella+Enterobacter (KE). Further, probe panel 400 includes a universal probe (EU) which is adapted to target substantially all microbial 16S rRNA sequences of eubacteria or similar microbes.

Certain species-specific probes have been selected particularly for blood sepsis management in a variety of settings.

In a preferred embodiment, capture probes as described above corresponding to each of the 12 targets of probe panel 400 may be arranged in a predetermined regular grid pattern on a substrate, with each predetermined position corresponding to the capture probe for a specific target microbe. A lysed clinical specimen may be contacted with the capture probe array and a mixture of detector probes corresponding to each of the 12 targets of probe panel 400. Signals from activated detector probes (those which have hybridized with target rRNA sequences) may be detected by detection means, such as a fluorescent camera system. The pattern of signal detection arranged in a regular grid pattern corresponding to the placement of the capture probes may be used for detection, thereby producing a unique output signal pattern corresponding to each target microbe.

### Assay Specificity

Some of the oligonucleotide probes of the present invention have relatively high species, genus and/or family specificity for each of the target microbes, which can help facilitate the detection of specific target microbes using specific, associate probe sets. Each probe set can be adapted to hybridize with high-accessibility regions of rRNA that are both highly specific for and highly conserved within each species or genus.

Shown in Table 5 is the accuracy rate of species or genus-specific identification using the probes of the present invention, when compared to standard identification methods known in the art. The assays performed for Table 2 were multiplexed with an array of capture probes anchored onto magnetic beads or an optical surface, with a mixture of detector probes.

TABLE 5
Urine Specimens and Isolates

| Species (Tests) | Accuracy (%) |
|---|---|
| E. Coli (123) | 98 |
| K. pneumoniae (78) | 100 |
| P. mirabilis (80) | 100 |
| P. aeruginosa (46) | 100 |
| Enterobacter spp. (33) | 97 |
| Other Enterobacteriaceae (39) | 100 |

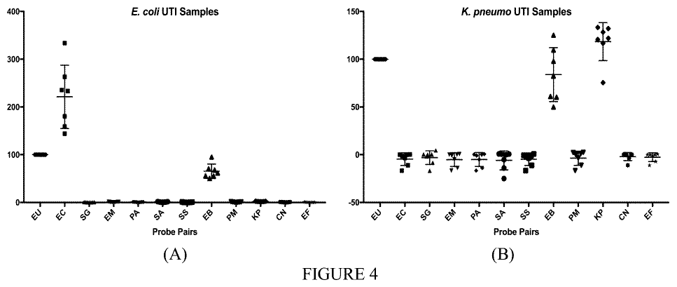

Referring now to FIG. 4, assays conducted using the multiplexed probe panel 300 of the present invention on clinical urine specimens from human patients with UTI, demonstrate the specificity of the probes. FIG. 4A shows resulting data from a clinical specimen containing E. coli, while FIG. 4B shows resulting data from a clinical specimen containing K. pneumoniae.

### Assay Sensitivity and Quality

The methods and probes of the present invention achieve a limit of detection of ≤10⁴ colony forming units per milliliter (cfu/ml), under a variety of platforms including on electrochemical sensor, ELISA, magnetic beads, and nitrocellulose array. This threshold is clinically useful for diagnosis of UTI. This threshold translates to approximately 0.1 pM of rRNA based on the number of copies per cell, as described in U.S. Provisional Application No. 62/671,380, filed May 14, 2018, the contents of which is incorporated herein by reference.

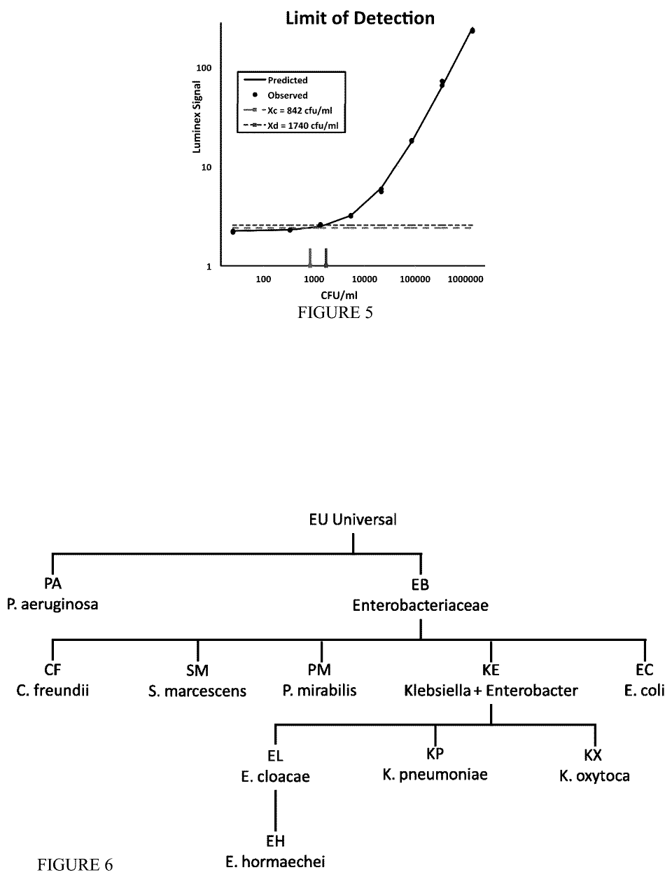

Shown in FIG. 5 is a graph of the signal obtained from a serially diluted clinical specimen containing E. coli cells, and the calculation of the critical limit (Xc) and limit of detection (Xd) obtained using an exemplary apparatus comprising a benchtop magnetic bead assay system (Luminex MAGPIX). This limit of detection has also been achieved for a nitrocellulose array implemented by the inventors.

The assay described herein is highly reproducible. Comparison of 30 positive and 30 negative results in a single 96 plate yielded extremely high Z-factor scores ranging from of 0.86 to 0.90.

### Automation

Preferably, some or all of the steps in the methods can be automated using suitable equipment and do not require a skilled laboratory technician or the like to process the specimens and/or interpret the results. In some embodiments described herein, the inputs for the analysis method is a generally “fresh”, unmodified specimen obtained from a patient, or an inoculum derived therefrom that is generated during conventional laboratory testing procedures, and the output of the method is an answer that is usable and/or understandable by a lay operator (i.e. not a trained lab technician). For example, the output may be in the form of text which represents which microbial species (if any) have been identified.

### Estimating Microbial Density in a Clinical Specimen

Identification of microbial species may be useful for the purpose of estimating the microbial density in a given clinical specimen, as the identity of the species may be used as an input in a method for quantification based on the relationship between rRNA and microbial density for a given species. For example, methods of estimating the microbial density are described in U.S. Provisional Application No. 62/671,380, filed May 14, 2018, the contents of which is incorporated herein by reference.

### Screening for Infection

Identification of microbial species may also be useful to determine the presence of pathogens in a clinical specimen which may be clinically relevant. Knowledge of the presence or absence of particular species may be of assistance to a clinician in indicating the presence of infection and the nature of the infection.

### Antimicrobial Susceptibility Testing

Identification of microbial species may further be useful for providing information to clinicians or lab technicians when conducting antimicrobial susceptibility testing. Knowledge of the microbial species present in a clinical sample may be used to further test or exclude certain known antimicrobial compounds. This, in turn, may assist in addressing the problem of antimicrobial resistance.

### Identification Systems

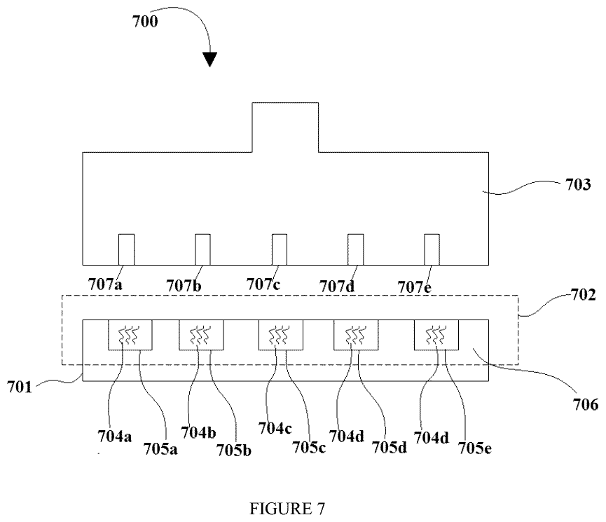

Optionally, one or more of the probe sets and/or probe panels described herein may be provided as part of a system for microbial identification that can be provided to users. The systems may optionally be tailored to common clinical uses, such as testing urine samples for UTI microbes, testing blood cultures for sepsis microbes and the like. One embodiment of a system 700 that may be used for microbial identification is provided in FIG. 7. Each system may include a carrier 701 (optionally a substrate, cartridge, disc, etc.) that can support a given probe panel. The carrier may take the form of a container that is pre-loaded with the probe panel 702 and into which the specimen being tested can be placed. For example, a user may be provided with a urine test system that includes a container suitable for holding a urine sample and that is preloaded with a probe panel including probe sets that are configured to target bacteria commonly associated with UTIs. The container may also include chambers for conducting the lysis, neutralization and other method steps—or such steps may be completed using a different container (possibly part of the same, single use system) or using other suitable techniques. In addition to the urine test systems, a user may be provided with sepsis test systems and other types of test systems focused on other relatively common front-line diagnostic tests.

Preferably, the container and probe panel are single-use and are configured to be disposed of when the identification testing is complete.

The system may also include a suitable detection apparatus 703, that is configured (and/or calibrated) to read the results of a given probe panel. For example, referring to FIG. 7, one example of the portions of one example of a system for microbial identification is provided where the system may include a carrier 701 that can house a probe panels 702, for example arranged in an array. As shown in FIG. 7, the carrier 701 may be configured to house at least two detection regions 705. In the example provided in FIG. 7, the contained is configured to have five detection regions (705a-e), however in other embodiments, the carrier 701 may be configured to have any number of detection regions. A plurality of first oligonucleotide probe sets 704a may be disposed in a first detection region 705a, and a plurality of second oligonucleotide probe sets 704b may disposed in the second detection region, and so on depending on the number detections regions on a specific container. Each plurality of oligonucleotide probe sets 704 may comprise one or more capture probes and one or more detector probes. The capture probes of each oligonucleotide probe set may be adapted to hybridize with a first target sequence of one of a plurality of rRNA molecules on a target microbe, while the detector probes of that same oligonucleotide probe set may comprise a detectably labeled oligonucleotide which may be adapted to hybridize with second target sequence of one of the plurality of rRNA molecules of the same target microbe. For example, the plurality of first oligonucleotide probe sets 704a in FIG. 7 may include a capture probes which is configured to Hybridge to a target sequence on an rRNA sequence of a target Gram negative bacteria. In this example, the plurality of first oligonucleotide probe sets 704a may also include detector probes which a include a detectably labeled oligonucleotide adapted to hybridize with a second target sequence on that same target Gram negative bacteria. As shown in FIG. 7, the system 700 may also include a detection apparatus 703. The detection apparatus may be configured to detect the first detector probes and the second detector probes in each of the detection regions 705 of the carrier 701. For example, when the system 700 is in use, a plurality of first hybridized complexes may be formed in the first detection region 705a, each including one first capture probe and one first detector probe and one of the plurality of rRNA molecules from a first target microbe, and a plurality of second hybridized complexes may be formed in the second detection region 705b, each including one second capture probe and one second detector probe and one of the plurality of rRNA molecules from a second target microbe, and the detection apparatus 703 may be configured to detect the first hybridized complexes in the first detection region 705a and to detect the second hybridized complexes in the second detection region 705b thereby indicating the presence of the first and second target microbes in the specimen.

As shown in FIG. 7, the pluralities of oligonucleotide probe sets (including their respective capture and detector probes) may be arranged in an array on a substrate 706. The substrate may comprise at least one of plastic, metal, glass, nitrocellulose, organic material and a magnetic-bead based platform.

As further shown in FIG. 7, the detection apparatus 703 may comprise one or more sensors 707. In some examples, the detection apparatus 703 may have a single sensor which is configured to detect a signal from any one of the hybridized complexes within any one of the detection regions 705 of the container. In other examples, as shown in FIG. 7, the detection apparatus 703 may include a number of sensors 707, where each sensor is configured to detect a signal from the first hybridized complexes within a corresponding detection region 705 on the container. For example, in the system 700 provided in FIG. 7, the detection apparatus includes five sensors (707a-e), where the first sensor 707a is configured to detect a signal from the hybridized complexes within the first detection region 705a, the second sensor 707b is configured to detect a signal from the hybridized complexes within the second detection region 705b, the third sensor 707c is configured to detect a signal from the hybridized complexes within the third detection region 705c, the fourth sensor 707d is configured to detect a signal from the hybridized complexes within the fourth detection region 705d, and the fifth sensor 705e is configured to detect a signal from the hybridized complexes within the fourth detection region 705e. In certain configurations, the one or more sensors 705a-d of the detection apparatus 703 may be configured to be operable simultaneously. In a particular preferred embodiment, each of the detection regions 705a may contain probe sets that may be used to detect a different target microbe than the probe sets in each of the other detection regions. For example, in the system 700 of FIG. 7, the probe sets in the first detection region 704a may be configured to indicate the presence of a first target microbe, and the probe sets in the second detection region 704b may be configured to indicate the presence of a second target microbe, and the probe sets in the third detection region 704c may be configured to indicate the presence of a third target microbe, and the probe sets in the fourth detection region 704d may be configured to indicate the presence of a fourth target microbe, and the probe sets in the fifth detection region 704e may be configured to indicate the presence of a fifth target microbe, where each of the first, second, third, fourth and fifth target microbes are different from one another.

What has been described above has been intended to be illustrative of the invention and non-limiting and it will be understood by the persons skilled in the art that other variants and modifications may be made without departing from the scope of the invention as defined by the claims appended hereto. The scope of the claims should not be limited by the preferred embodiments and examples, but should be given the broadest interpretation consistent with the description as a whole.

1. An oligonucleotide probe set for use in identifying a first target microbe in a specimen, wherein the oligonucleotide probe set comprises a capture probe and a detector probe, the capture probe and detector probe each being adapted to selectively hybridize to a first target sequence of rRNA molecules released from the first target microbe.

2. The oligonucleotide probe set of claim 1, wherein the oligonucleotide probe set is adapted to selectively bind to rRNA molecules released from a pre-determined microorganism, preferably a microorganism selected from the group consisting of Escherichia coli (EC), Klebsiella pneumoniae (KP), Proteus mirabilis (PM), Pseudomonas aeruginosa (PA), Staphylococcus saprophyticus (SS), Staphylococcus aureus (SA), Streptococcus agalactiae (GB), Enterococcus faecium (EM), Candida albicans (CA), Enterobacteriaceae (EB), Enterococci (EF), Candida species (CN), Citrobacter freundii (CF), Serratia marcescens (SM), Enterobacter cloacae (EL), Enterobacter hormaechei (EH), and Klebsiella oxytoca (KX), Klebsiella aerogenes (KA), Morganella morganii (MM), Acinetobacter baumanii (AB), Streptococcus pyogenes (SY), Streptococcus pneumoniae (SP), Streptococcus viridans (SV), Stenotrophomonas maltophilia (XM), Staphylococcus epidermidis (SE) and combinations of any two or more of these.

3. The oligonucleotide probe set of claim 1, wherein the oligonucleotide probe set is adapted to hybridize with rRNA molecules released from substantially all eubacteria (EU) or similar microbes.

4. A probe panel disposed on a substrate, the probe panel comprising a plurality of detection regions, each detection region comprising an oligonucleotide probe set configured to selectively bind to RNA molecules released from a pre-determined microorganism, each detection region comprising a different oligonucleotide probe set.

5. The probe panel of claim 4, wherein each oligonucleotide probe set comprises a first capture probe having an oligonucleotide adapted to hybridize with a first target sequence of the rRNA molecules of the pre-determined microorganism, and a first detector probe having a detectably labeled oligonucleotide adapted to hybridize with a second sequence of the rRNA molecules of the pre-determined microorganism.

6. The probe panel of claim 4, wherein the pre-determined microorganism is selected from the group consisting of: Escherichia coli (EC), Klebsiella pneumoniae (KP), Proteus mirabilis (PM), Pseudomonas aeruginosa (PA), Staphylococcus saprophyticus (SS), Staphylococcus aureus (SA), Streptococcus agalactiae (GB), Enterococcus faecium (EM), Candida albicans (CA), Enterobacteriaceae (EB), Enterococci (EF), Candida species (CN), Citrobacter freundii (CF), Serratia marcescens (SM), Enterobacter cloacae (EL), Enterobacter hormaechei (EH), and Klebsiella oxytoca (KX), Klebsiella aerogenes (KA), Morganella morganii (MM), Acinetobacter baumanii (AB), Streptococcus pyogenes (SY), Streptococcus pneumoniae (SP), Streptococcus viridans (SV), Stenotrophomonas maltophilia (XM), Staphylococcus epidermidis (SE) and any combination of two or more of these.

7. The probe panel of claim 4, wherein the substrate comprises at least one of plastic, metal, glass, nitrocellulose, organic material and a magnetic-bead based platform.

8. The oligonucleotide probe set of claim 1, wherein the oligonucleotide probe set further comprises a helper probe adapted to hybridize with a helper sequence of the capture probe to help immobilize the capture probe.

9. The oligonucleotide probe set of claim 1, wherein the oligonucleotide probe set is adapted to hybridize 16S rRNA sequences.

10. The oligonucleotide probe set of claim 1, wherein the oligonucleotide probe set is adapted to hybridize 23S rRNA sequences.

11. The probe panel of claim 4, wherein the number of detection regions is in the range of between 3 and 100.

12. The probe panel of claim 4, wherein the number of detection regions is in the range of between 5 and 50.

13. The probe panel of claim 4, wherein the number of detection regions is in the range of between 10 and 25.

14. A system for identifying at least two microbes in a specimen, the system comprising:
(a) the probe panel of claim 4; and
(b) a detection apparatus configured to detect hybridized complexes present in any detection regions of the probe panel.

15. The system of claim 14, wherein the detection apparatus comprises a plurality of sensors, each sensor associated with a single detection region and configured to detect a signal from a hybridized complex when present in the single detection region.

16. The system of claim 15, wherein the plurality of sensors are operable simultaneously.

17. The system of claim 14, wherein the detection apparatus comprises a single sensor, moveable to be associated with each detection region and configured to detect a signal from a hybridized complex when present in a detection region proximal to the single sensor.

18. The system of claim 14, wherein the specimen is selected from the group consisting of urine, blood, serum, plasma, saliva, tears, gastric fluids, digestive fluids, stool, mucus, sputum, sweat, earwax, oil, semen, vaginal fluid, glandular secretion, breast milk, synovial fluid, pleural fluid, lymph fluid, amniotic fluid, feces, cerebrospinal fluid, wounds, burns, tissue homogenates and an inoculum derived therefrom that is generated during conventional laboratory testing procedures.
